(19) United States
(12) Patent Application Publication
(10) Pub. No.: US 2022/0220433 A1
(43) Pub. Date: Jul. 14, 2022

(54) METHOD FOR OPTIMIZING A FERMENTATION PROCESS

(71) Applicant: Unibio A/S, Roskilde (DK)

(72) Inventors: Subir Kumar Nandy, Kongens Lyngby (DK); Leander Petersen, Troy, NY (US); Ib Christensen, Allerød (DK)

(21) Appl. No.: 17/616,446

(22) PCT Filed: Jun. 3, 2020
(86) PCT No.: PCT/EP2020/065347

§ 371 Date: Dec. 3, 2021

(30) Foreign Application Priority Data
Jun. 7, 2019 (DK) PA 2019 00697
Oct. 31, 2019 (DK) PA 2019 01279

(51) Int. Cl. C12N 1/30; C12N 1/20; A23K 10/10; A23L 29/00

(52) U.S. Cl. CPC C12N 1/30; C12N 1/20; C12N 2500/02; A23L 29/065; A23K 10/10

(57) ABSTRACT

The present invention relates to a process for cultivating one or more microorganisms capable of metabolising methane, the process comprises the steps of: (i) adding a fermentation medium to a fermentation reactor; (ii) adding the one or more microorganism to a fermentation reactor, providing an inoculated fermentation medium, wherein the one or more microorganism does not include a recombinant microorganism; (iii) adding a C1-C5 carbon source, e.g. methane, to the fermentation reactor and/or the inoculated fermentation medium during the fermentation of the one or more microorganisms; and (iv) optionally, adding oxygen to the fermentation reactor and/or the inoculated fermentation medium during the fermentation of the one or more microorganisms, wherein the oxygen is added to the fermentation reactor and/or the inoculated fermentation medium to provide a content of undissolved oxygen in the fermentation reactor and/or a content of gaseous oxygen in an exhaust gas is at most 10% (vol/vol).

### THE TECHNICAL FIELD OF THE INVENTION

The present invention relates to a fermentation reactor and a fermentation process. In particular, the present invention relates to a fermentation reactor comprising gas sensors and a process for the optimised fermentation of methanotrophic or methylotrophic microorganisms.

### BACKGROUND OF THE INVENTION

During traditional fermentation of microorganisms capable of metabolising methane, the addition of methane gas and oxygen gas is very important in order to promote growth of the microorganism. The gases are injected into the fermentation medium and subjected to various mixing steps in order to dissolve as much of the gases in the fermentation medium as possible and thereby making it available for propagation and growth of the microorganism to be cultivated. As it is impossible to dissolve all the gas introduced into the fermenter and as the phase equilibrium between liquid and gas results in a certain amount of the methane gas and the oxygen gas to be undissolved gasses which are to be discharged from the fermentation reactor.

The presence of undissolved oxygen may result in several disadvantages, whereby the process and the level of undissolved of oxygen needs to be very strictly controlled during fermentation, and preferably avoided.

One challenge with the undissolved gasses, comprising oxygen and methane, is a risk of fire and explosion either at the exit or even inside the fermentation reactor.

Thus, under the wrong circumstances, the mixture of oxygen gases and methane gases can cause an explosion hazard, as represented in FIG. 1 as the gray area.

Fire can start due to the presence of a fuel, an oxidant and an ignition source. Oxygen in air is normally used as an oxidant. Therefore, fire can start in the presence of a certain concentration of both fuel and oxidant. FIG. 1 is a typical triangle diagram (an explosion triangle) for a flammable mixture of oxygen and methane and which also comprises nitrogen, however, the nitrogen acts as an inert gas (a passive agent). This means, when the nitrogen concentration increases, the flammability range decreases, and no ignition were observed more than 84% (see FIG. 1). Nitrogen can also be exchanged to Carbon-di-oxide (CO₂) where no ignition point observed when the CO₂ content exceeds 73%.

The upper explosion limit (UEL) and the lower explosion limit (LEL) are the most important properties of flammable gases. Most flammable materials demonstrate a flammability zone (the shaded gray area in FIG. 1), that is, a region within which the mixture (in FIG. 1, the mixture of methane and oxygen) is flammable at all concentrations. The current operation protocol for the U-loop technology includes overfeeding or purging with air/oxygen to stay in the area “below” the lower explosion limit. In present process oxygen is added to the reactor providing about 2.5% methane in the exhaust gas to ensure not to be in the flammability zone/explosion area. This strategy results in high consumption and loss of oxygen and low methane concentrations in the exhaust gas which is vented to the atmosphere and lost.

A standard stoichiometry, as shown in formulas (1) and (2) below, illustrates why the high concentrations of oxygen relative to the concentration of methane has been used. From the standard stoichiometric point of view the demand of oxygen is higher than the demand for methane in order to provide the desired biomass product, where methane react with oxygen under nitrogen source.

Standard stoichiometry equation 1 is demonstrated on nitrate-based stoichiometry and in standard stoichiometry equation 2, ammonia is used as a nitrogen source.

The stoichiometry are as follows:

$$\begin{matrix}{CH}_{4} + 1.22 {O}_{2} + 0.104 {NaNO}_{3} \to 0.52 Biomass + 0.48 {CO}_{2} + 1.532 {H}_{2} O \\ and & ( 1 ) \\ {CH}_{4} + 1.45 {O}_{2} + 0.104 {NH}_{3} \to 0.52 Biomass + 0.48 {CO}_{2} + 1.69 {H}_{2} O & ( 2 )\end{matrix}$$

From the standard stoichiometry equations 1 and 2, using one mol of methane more mol of oxygen are used in the process (1.22 mol of oxygen when nitrate as a nitrogen source and 1.45 mol of oxygen when ammonia is used as a nitrogen source).

Therefore, fermentation processes described today use more oxygen than actually necessary, based on this theoretical stoichiometry, and this makes the fermentation process a more dangerous process in terms of flammability issues and it also provides a less environment friendly and a more costly process.

Hence, in order to limit the risk of fire and/or explosion of the conventional fermentation reactors, much higher amounts of oxygen gas than methane gas is added to the fermentation medium and the process is controlled in order to avoid more than 5% undissolved methane in the exhaust gas, and thereby reducing the risk of fire and explosion. Hence, the amount of oxygen (dissolved and undissolved) in the fermentation reactor is significantly higher than the amount of methane (dissolved and undissolved).

Another disadvantage of having this surplus of oxygen relative to the amount of methane (dissolved and undissolved) in the fermentation reactor is that it may create a challenge in respect of the stability and functionality of the fermentation process. If this challenge is not handled correctly, there is a significant increased risk of poisoning the fermentation medium with nitrite which may be formed when ammonia (which is added as nitrogen source during the fermentation process illustrated in standard stoichiometry equation 2) is oxidized under nitrification process, which is the rate limiting step of nitrification. During nitrification, the microorganism is under extreme stress and nitrite may be formed. Since nitrite cannot be converted totally into nitrate and will stay in the fermenter, the fermentation process must be stopped, and the fermentation medium is discarded and a new batch must be started. This process is called “the route of death” of the fermentation and is feared by all single cell protein produces.

Ammonia is one of the cheapest sources of nitrogen and has become the standard or the preferred choice of nitrogen source used during the fermentation processes. However, ammonia forms nitrite under any stressed conditions and bacteria cannot grow under certain nitrite levels. Nitrite forms bacteria inhibiting growth which is a condition that is not to be saved and which results in the so called “route of death”.

Hence, an improved fermentation process and an improved fermentation reactor would be advantageous, and in particular it would be advantageous to provide a fermentation reactor and a fermentation process being safer, more environmental friendly, more cost-efficient, more simple, more effective, more reliable, reducing the risk of failure, and without compromising the cost challenge in the industry.

### SUMMARY OF THE INVENTION

Thus, an object of the present invention relates to an improved fermentation reactor and an improved fermentation process for cultivating one or more methanotrophic or one or more methylotrophic microorganisms.

In particular, it is an object of the present invention to provide a fermentation reactor and a fermentation process that solves the above-mentioned problems of the prior art with safety, cost, simplicity, harmful to the environment, in-efficiency, being un-reliable, the risk of failure (e.g. due to “the route of death”).

Thus, one aspect of the present invention relates to a process for cultivating one or more microorganisms capable of metabolising methane, the process comprises the steps of:
(i) adding a fermentation medium to a fermentation reactor;
(ii) adding the one or more microorganism to a fermentation reactor, providing an inoculated fermentation medium, wherein the one or more microorganism does not include a recombinant microorganism;
(iii) adding a C1-C5 carbon source, e.g. methane, to the fermentation reactor and/or the inoculated fermentation medium during the fermentation of the one or more microorganisms; and
(iv) optionally, adding oxygen to the fermentation reactor and/or the inoculated fermentation medium during the fermentation of the one or more microorganisms,

wherein the oxygen is added to the fermentation reactor and/or the inoculated fermentation medium to provide a content of undissolved oxygen in the fermentation reactor and/or a content of gaseous oxygen in an exhaust gas is at most 10% (vol/vol).

A further aspect of the present invention relates to a process for cultivating one or more microorganisms capable of metabolising methane, the process comprises the steps of:
(i) adding a fermentation medium to a fermentation reactor;
(ii) adding the one or more microorganism to a fermentation reactor, providing an inoculated fermentation medium, wherein the one or more microorganism does not include a recombinant microorganism;
(iii) adding a C1-C5 carbon source, e.g. methane, to the fermentation reactor and/or the inoculated fermentation medium during the fermentation of the one or more microorganisms; and
(iv) optionally, adding oxygen to the fermentation reactor and/or the inoculated fermentation medium during the fermentation of the one or more microorganisms,

wherein the process is a semi-aerobic fermentation process or an anaerobic fermentation process wherein the content of undissolved oxygen in the fermentation reactor or the content of gaseous oxygen present in an exhaust gas may be at most 5% (vol/vol).

Yet an aspect of the present invention relates to a fermentation reactor for fermenting one or more microorganisms, the fermentation reactor comprising at least one inlet for introducing a C1-C5 carbon source, such as methane, into the fermentation reactor and one or more sensors, wherein the one or more sensors are one or more sensors for determining the concentration of undissolved and/or released gases in the fermentation reactor, wherein the undissolved and/or released gases is oxygen.

Another aspect of the present invention relates to a fermentation reactor for fermenting one or more microorganisms, the fermentation reactor comprising at least one inlet for introducing a C1-C5 carbon source into the fermentation reactor and one or more sensors, wherein the one or more sensors are one or more sensors for determining the concentration of undissolved and/or released gases in the fermentation reactor.

A further aspect of the present invention relates to a fermentation reactor for fermenting one or more microorganisms, the fermentation reactor comprising at least one gas inlet for introducing a methane gas into the fermentation reactor; and a vent for discharging undissolved and/or released gas(es) from the fermentation reactor, and one or more sensors, wherein the one or more sensors are one or more sensors for determining the concentration of undissolved and/or released gases in the fermentation reactor.

Yet a further aspect of the present invention relates to a biomass material obtainable by the process according to the present invention, preferably the biomass material comprises at least 25 genes having a reduced transcript level of at least 25% (w/w).

An even further aspect of the present invention relates to a feed product for an animal; a fish feed product; or a human food product comprising the biomass material according to the present invention.

Yet another aspect of the present invention relate to a system for reducing the environmental impact and/or the energy consumption of a fermentation process when cultivating one or more microorganisms capable of metabolising methane, the system comprising a fermentation reactor according to the present invention, and an exhaust gas pipe connecting the vent for discharging undissolved and/or released gas(es) from the fermentation reactor with a generator, for power production; a heating device, for generating heat; and/or cleaning unit for recycling methane to the fermentation reactor.

### BRIEF DESCRIPTION OF THE FIGURES

FIG. 1 shows the Explosion Triangle. The sides of the Explosion Triangle indicate how many % of the total gas composition is composed of each species. The grey area is the ratio where the gas composition becomes flammable, and where the explosion risk is high,

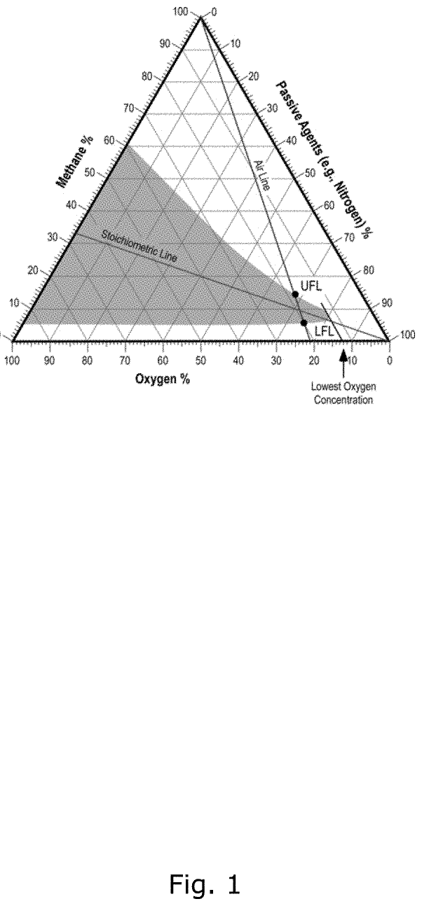

FIG. 2 shows the growth of methanotrophs (M. capsulatus) under anaerobic conditions for 92 hours at continuous fermentation. After fermenting for 68 hours the supply of oxygen was stopped and the continuous growth of the biomass may be followed by the reduction in the acid level (indicated by triangles); the NO₂⁻ level (indicated by squares) and the NO₃⁻ level (indicated by circles), which all drops significantly over a period of 24 hours (from 68 hours from the start of continuous fermentation to 92 hours). Hence, anaerobic fermentation of methanotrophs, such as M. capsulatus may be performed without the supply of gaseous oxygen.

The present invention will now be described in more detail in the following.

### DETAILED DESCRIPTION OF THE INVENTION

Accordingly, the present invention relates to a fermentation reactor and a fermentation process for the cultivation of a microorganism capable of metabolizing C1-C5 compounds, like methane, such as a methanotroph or a methylotroph, such as a microorganism selected from the family Methylococcaceae or Methylocystaceae, which may be fermented under reduced oxygen level; reduced oxygen/methane ratio, or preferably under anaerobic conditions, and which process show to be more reliable, has a lower risk of failure, more efficient and less harmful to the environment and at the same time more economical interesting relative to conventional processes for fermenting microorganisms capable of metabolizing C1-C5 compounds, like methane.

Hence, a preferred embodiment of the present invention relates to a process for cultivating one or more microorganisms capable of metabolising methane, the process comprises the steps of:
(i) adding a fermentation medium to a fermentation reactor;
(ii) adding the one or more microorganism to a fermentation reactor, providing an inoculated fermentation medium;
(iii) adding a C1-C5 carbon source, e.g. methane, to the fermentation reactor and/or the inoculated fermentation medium during the fermentation of the one or more microorganisms; and
(iv) optionally, adding oxygen to the fermentation reactor and/or the inoculated fermentation medium during the fermentation of the one or more microorganisms,

wherein the process is a semi-aerobic fermentation process or an anaerobic fermentation process.

Preferably, the one or more microorganism may be one or more aerobic microorganism.

The one or more aerobic microorganism may preferably, be selected from one or more aerobic methanotrophic microorganism and/or or one or more aerobic methylotrophic microorganism.

The one or more aerobic methanotrophic microorganism or one or more aerobic methylotrophic microorganism may be one or more aerobic methanotrophic bacteria and/or one or more aerobic methylotrophic bacteria.

In an embodiment of the present invention the one or more aerobic methanotrophic bacteria may be selected from a Methylococcus. Preferably, M. capsulatus.

In a further embodiment of the present invention the one or more aerobic methanotrophic bacteria may comprises a combination of M. capsulatus (preferably NCIMB 11132); A. acidovorans (preferably NCIMB 13287); B. firmus (preferably NCIMB 13289); and A. danicus (preferably NCIMB 13288).

In a preferred embodiment of the present invention the one or more microorganism does not include a recombinant microorganism.

In the context of the present invention the term “recombinant microorganism” relates to a genetically modified organism (GMO) whose genetic material has been altered using plasmids, deletion of existing genes; or other genetic engineering techniques. The recombinant microorganism may be considered in contrast to genetic alterations that occur naturally in the microorganism, e.g. by mating and/or natural mutation.

Preferably, the one or more microorganism may be one or more naturally occurring microorganism.

In the context of the present invention the term “naturally occurring microorganism” relates to a microorganism whose genetic material has not been altered using genetic engineering techniques. Natural modifications or alterations in the genetic material of a microorganism may be considered to be covered by the term “naturally occurring microorganism”.

In an embodiment of the present invention the semi-aerobic fermentation process may be a fermentation process wherein the content of undissolved oxygen in the fermentation reactor or the content of gaseous oxygen present in an exhaust gas may be at most 10% (vol/vol), such as at most 8% (vol/vol), e.g. at most 6% (vol/vol), such as at most 4% (vol/vol), e.g. at most 2% (vol/vol), such as at most 1% (vol/vol), e.g. at most 0.5% (vol/vol), such as 0% (vol/vol).

In the context of the present invention, the term “semi-aerobic fermentation” relates to a fermentation process wherein oxygen is added to the fermentation process and/or to the fermentation reactor, however, the content of oxygen added to the fermentation process and/or to the fermentation reactor, is smaller than the content of the C1-C5 carbon source, e.g. methane, added.

A further preferred embodiment of the present invention relates to a process for cultivating one or more microorganisms capable of metabolising methane, the process comprises the steps of:
(i) adding a fermentation medium to a fermentation reactor;
(ii) adding the one or more microorganism to a fermentation reactor, providing an inoculated fermentation medium;
(iii) adding a C1-C5 carbon source, e.g. methane. to the fermentation reactor and/or the inoculated fermentation medium during the fermentation of the one or more microorganisms; and
(iv) optionally, adding oxygen to the fermentation reactor and/or the inoculated fermentation medium during the fermentation of the one or more microorganisms,

wherein the oxygen is added to the fermentation reactor and/or the inoculated fermentation medium to provide a content of undissolved oxygen in the fermentation reactor and/or a content of gaseous oxygen in an exhaust gas is at most 10% (vol/vol).

In the present context, the C1-C5 carbon source may be added to the fermentation reactor. The C1-C5 carbon source may be a C1-C3 carbon source. Preferably the C1-C5 carbon source may comprise an alkane or an aldehyde. The alkane may preferably be a C1 compound and/or a C1 alkane and/or a C1 aldehyde or a derivative thereof.

Preferably the C1 compound and/or the C1 alkane may be methane, methanol, natural gas, biogas, syngas or any combination hereof. Preferably, the C1 aldehyde may be formaldehyde or a derivative thereof. Even more preferably, the C1 compound and/or the C1 alkane may be methane.

The C1-C5 carbon source added in step (iii) may be in liquid form or gaseous form. In an embodiment of the present invention the C1-C5 carbon source, e.g. methane, as added in step (iii) of the process according to the present invention may preferably be gaseous methane.

In a further embodiment of the present invention the oxygen as added in step (iv) of the process according to the present invention may be gaseous oxygen or liquid oxygen. Preferably, the oxygen may be provided as atmospheric air, pure oxygen, or air enriched with oxygen.

In semi-aerobic fermentation process the undissolved gas (and the exhaust gas) may comprise gasses in addition to methane gas and oxygen gas. Even other gases may be present the ratio between C1-C5 carbon source, e.g. methane, and oxygen may be important.

Hence, in an embodiment of the present invention the ratio between C1-C5 carbon source, e.g. methane, and oxygen in the undissolved gas, and/or in the exhaust gas may be at least 5:1 (vol C1-C5 carbon source/vol oxygen), such as at least 6:1 (vol C1-C5 carbon source/vol oxygen), e.g. at least 7:1 (vol C1-C5 carbon source/vol oxygen), such as at least 8:1 (vol C1-C5 carbon source/vol oxygen), e.g. at least 9:1 (vol C1-C5 carbon source/vol oxygen), such as at least 10:1 (vol C1-C5 carbon source/vol oxygen), e.g. at least 15:1 (vol C1-C5 carbon source/vol oxygen), such as at least 20:1 (vol C1-C5 carbon source/vol oxygen), e.g. at least 25:1 (vol C1-C5 carbon source/vol oxygen), such as at least 30:1 (vol C1-C5 carbon source/vol oxygen), e.g. at least 35:1 (vol C1-C5 carbon source/vol oxygen).

During fermentation it may be important to differentiate between dissolved gasses and undissolved gasses, since only the dissolved gases are consumable to the microorganisms. Undissolved gasses, like undissolved C1-C5 carbon source, like methane, and/or undissolved oxygen, which are to be separated in the exhaust gas and may be wasted and may increase the risk of explosion, as mentioned.

In the present context, the term “dissolved” relates to gas which is absorbed by the fermentation medium, in the present context, gas which has been absorbed by the fermentation medium and become available for consumption by the microorganisms to be cultivated. In contrast to the dissolved gas, there are the undissolved gas. In the present context, the term “undissolved” relates to gas which has not been absorbed by the fermentation medium and will not be available to be consumed by the microorganisms to be cultivated.

In a further embodiment of the present invention the fermentation process may be a semi-aerobic fermentation process having 0% (vol/vol) undissolved oxygen and more than 1% (vol/vol) dissolved oxygen relative to the fermentation medium, such as more than 3% (vol/vol) dissolved oxygen, e.g. more than 5% (vol/vol) dissolved oxygen; such as more than 3% (vol/vol) dissolved oxygen; e.g. more than 10% (vol/vol) dissolved oxygen; such as more than 15% (vol/vol) dissolved oxygen; e.g. more than 20% (vol/vol) dissolved oxygen; such as more than 25% (vol/vol) dissolved oxygen, e.g. in the range of 1-50% (vol/vol) dissolved oxygen; such as in the range of 5-25% (vol/vol) dissolved oxygen. The fermentation reactor may comprise an inlet for introducing CO₂ into the fermentation medium. In an embodiment of the present invention CO₂ may be added to the fermentation reactor and/or to the fermentation medium. The addition of CO₂ may be a continuous injected into the fermentation medium. Preferably, the amount of CO₂ injected into the fermentation medium is at least 0.001 L/min/L fermentation medium, such as at least 0.005 L/min/L fermentation medium, e.g. at least 0.01 L/min/L fermentation medium, such as at least 0.05 L/min/L fermentation medium, e.g. at least 0.1 L/min/L fermentation medium, such as at least 0.13 L/min/L fermentation medium, e.g. at least 0.15 L/min/L fermentation medium, such as at least 0.2 L/min/L fermentation medium, e.g. at least 0.25 L/min/L fermentation medium, such as at least 0.3 L/min/L fermentation medium, e.g. at least 0.4 L/min/L fermentation medium, such as at least 0.5 L/min/L fermentation medium, e.g. at least 0.60 L/min/L fermentation medium, such as at least 0.7 L/min/L fermentation medium, e.g. at least 0.75 L/min/L fermentation medium.

In an embodiment of the present invention, the content of undissolved CO₂ in the fermentation reactor or the content of gaseous CO₂ present in the exhaust gas is above 2.5% (vol/vol), such as at least 4% (vol/vol), e.g. at least 5% (vol/vol), such as at least 7.5% (vol/vol), e.g. at least 10% (vol/vol), such as at least 12.5% (vol/vol), e.g. at least 15% (vol/vol), such as at least 20% (vol/vol), e.g. at least 30% (vol/vol), such as at least 40% (vol/vol), and/or the content of undissolved CO₂ is less than 75% (vol/vol), such as less than 70% (vol/vol), e.g. less than 60% (vol/vol), such as less than 50% (vol/vol), e.g. less than 40% (vol/vol), such as less than 30% (vol/vol), e.g. less than 20% (vol/vol), such as less than 10% (vol/vol), e.g. less than 5% (vol/vol).

In yet an embodiment of the present invention the mole ratio between C1-C5 carbon source, e.g. methane, and CO₂ dissolved in the fermentation medium is about 1:1 mole ratio, such as about 0.525:0.475 mole ratio.

In a further embodiment of the present invention the undissolved gas may be in gaseous phase.

When the undissolved gas includes methane or methane and oxygen there is at some point a non-optimal fermentation conditions, as e.g. insufficient amount of the gases is dissolved in the fermentation medium which may affect growth of the microorganism which may grow slowly or grow may stop; or there may be a too extensive addition of gasses to the fermentation reactor which may affect the costs and the productivity of the fermentation process, as well as there may become a security risk as in accordance with the Explosion Triangle, where certain concentrations of methane and oxygen is highly flammable and hence constitute an explosion risk.

Hence, there may be a need for a constant regulation of the process in order to optimise the fermentation condition and at the same time reducing the flammability/explosion risk.

An equilibrium between gas in the undissolved phase and the dissolved phase exists. This equilibrium may be manipulated in one way to increase the amount of dissolved gas in the fermentation medium and the equilibrium may be manipulated in another way to increase undissolved gas to allow unconsumed methane and/or unconsumed oxygen, and/or CO₂ generated by the growth of the microorganism and/or CO₂ added, to be released from the fermentation reactor. Hence, it may be impossible to only add just enough methane and/or oxygen to obtain optimal fermentation conditions and avoid having methane and/or oxygen in the undissolved phase.

The methane and/or oxygen show a tendency to favour the undissolved phase and in order to improve the content of oxygen and methane to be dissolved in the inoculated fermentation medium constant mixing in the fermentation reactor may be provided.

To avoid having a undissolved gas phase comprising methane and oxygen in flammable amounts (ratio), and the thus having the risk of explosion, excessive amounts of oxygen is traditionally added to the fermentation reactor and/or to the fermentation medium to ensure not to be in in flammable amounts (ratio) of the gases. Hence, traditionally fermentation processes when cultivating microorganisms capable of metabolising methane have been controlled by regulating the amount/concentration of undissolved oxygen.

The inventors of the present invention surprisingly found that instead of controlling and regulating the fermentation process based on an excessive content of oxygen in the exhaust gas, the process may be preferably be controlled by providing an excessive content of methane in the exhaust gas, which methane may optionally be recycled or used as energy source in e.g. downstream processes, and in this way provide a process which is more reliable, has a lower risk of failure, more efficient and less harmful to the environment and at the same time more economical interesting relative to conventional processes for fermenting microorganisms capable of metabolizing methane. The exhaust gas may be cleaned to provide pure, or substantially pure methane gas, e.g. by stripping CO₂ and/or O₂ (if present) from the exhaust gas, before recycling the methane to the reactor or before using it as energy source.

In an embodiment of the present invention the methane present in the exhaust gas and/or oxygen is present in the exhaust gas may be recycled to the fermenter. Preferably, CO₂ and/or sulphur compounds present in the exhaust gas may be partly of fully removed from the methane and/or oxygen before being reinjected the or recycled to the fermenter.

Further advantages of controlling and regulating the fermentation process based on an excessive content of C1-C5 carbon source, e.g. methane, is that by using a higher content of C1-C5 carbon source, e.g. methane, to a lower level of explosion (LEL) of oxygen decreases the flammability and explosive issues of the process. Furthermore, by adding more C1-C5 carbon source, e.g. methane, according to the present invention also decreases the possibility of nitrite formation, which is a problem in the presence of excessive amounts of oxygen and that may result in failure of the fermentation process, due to the route of death of the culture.

Hence, in an embodiment of the present invention, the amount of C1-C5 carbon source, e.g. methane, and/or oxygen added in steps (iii) and/or step (iv) may be controlled by the content of undissolved and/or released C1-C5 carbon source, e.g. methane, and/or oxygen in an exhaust gas.

The methane obtained from the exhaust gas may be recycled to the fermentation reactor or used for energy consumption generating e.g. electricity and/or heating for one or more downstream processes, such as a dryer, e.g. a flash dryer; a separator, e.g. a centrifuge; and/or other down-steam processing devices for the harvested product.

In an embodiment of the present invention the addition of C1-C5 carbon source, e.g. methane, (step iii) includes a continuous addition of C1-C5 carbon source, e.g. methane, to the fermentation reactor and/or to the inoculated fermentation medium.

In a further embodiment of the present invention the addition of oxygen (step iv) includes a continuous addition of oxygen to the fermentation reactor and/or to the inoculated fermentation medium.

The continuous addition of oxygen and/or C1-C5 carbon source, e.g. methane, may be controlled by the content of undissolved and/or released C1-C5 carbon source, e.g. methane, and/or oxygen in an exhaust gas or by the content of undissolved and/or released C1-C5 carbon source, e.g. methane, and/or oxygen in the inoculated fermentation medium.

In a preferred embodiment of the present invention the fermentation process may be a semi-aerobic fermentation process or a semi-anaerobic fermentation process.

Preferably, the “semi-aerobic fermentation process” or the “semi-anaerobic fermentation process” may be used interchangeably, and may relate to a fermentation process where the content of undissolved oxygen in the fermentation reactor and/or the content of undissolved (gaseous) oxygen present in the exhaust gas is equal or lower than or reduced to be equal or lower than the content of the C1-C5 carbon source, e.g. methane.

In another embodiment of the present invention the “semi-aerobic fermentation process” or the “semi-anaerobic fermentation process” may be used interchangeably, and may relate to a fermentation process wherein the content of oxygen dissolved in the fermentation medium in the fermentation reactor, is equal or smaller than the content of the C1-C5 carbon source, e.g. methane.

In yet an embodiment of the present invention the “semi-aerobic fermentation process” or the “semi-anaerobic fermentation process” may be used interchangeably, and may relate to a fermentation process wherein the content of oxygen added to the fermentation reactor is equal or smaller than the content of the C1-C5 carbon source, e.g. methane added to the fermentation reactor.

In a further embodiment of the present invention, the “semi-aerobic fermentation process” or the “semi-anaerobic fermentation process” may be used interchangeably, and may relate to:
a fermentation process where the content of undissolved oxygen in the fermentation reactor and/or the content of undissolved (gaseous) oxygen present in the exhaust gas is equal or lower than or reduced to be equal or lower than the content of the C1-C5 carbon source, e.g. methane; and/or
a fermentation process wherein the content of oxygen dissolved in the fermentation medium in the fermentation reactor, is equal or smaller than the content of the C1-C5 carbon source, e.g. methane; and/or
a fermentation process wherein the content of oxygen added to the fermentation reactor is equal or smaller than the content of the C1-C5 carbon source, e.g. methane added to the fermentation reactor.

In an embodiment of the present invention the semi-aerobic fermentation process may be a fermentation process wherein the content of undissolved oxygen in the fermentation reactor or the content of gaseous oxygen present in an exhaust gas may be at most 10% (vol/vol), such as at most 8% (vol/vol), e.g. at most 6% (vol/vol), such as at most 4% (vol/vol), e.g. at most 2% (vol/vol), such as at most 1% (vol/vol), e.g. at most 0.5% (vol/vol), such as 0% (vol/vol).

Preferably the content of undissolved oxygen in the fermentation reactor or the content of gaseous oxygen present in an exhaust gas may be at most 10% (vol/vol), such as at most 8% (vol/vol), e.g. at most 6% (vol/vol), such as at most 4% (vol/vol), e.g. at most 2% (vol/vol), such as at most 1% (vol/vol), e.g. at most 0.5% (vol/vol), such as 0% (vol/vol).

In a further embodiment of the present invention the fermentation process may be an anaerobic fermentation process.

In the context of the present invention the term “anaerobic fermentation process” relates to a fermentation process where oxygen, preferably gaseous oxygen or liquid oxygen, is not added to the fermentation reactor.

The fermentation process according to the present invention may be started as an anaerobic fermentation process; or as an aerobic or a semi-aerobic fermentation process and followed by an anaerobic fermentation process where the supply of oxygen, gaseous oxygen or liquid oxygen, is stopped.

In an embodiment of the present invention the fermentation process may initially be a semi-aerobic fermentation process followed by an anaerobic fermentation process.

The fermentation process according to the present invention may be an aerobic or a semi-aerobic fermentation process and followed by an anaerobic fermentation process where the supply of oxygen, preferably gaseous oxygen, may be stopped. Preferably, the supply of oxygen, preferably gaseous oxygen, for the anaerobic fermentation process may be stopped for a period of at least 2 hours, such as at least 5 hours, e.g. at least 10 hours, such as at least 20 hours, e.g. at least 24 hours, such as at least 30 hours, e.g. at least 35 hours, such as at least 40 hours, e.g. at least 50 hours, such as at least 60 hours, e.g. at least 70 hours.

In yet an embodiment of the present invention the process of the present invention does not involve addition of gaseous oxygen to the fermentation medium.

In yet an embodiment of the present invention the process of the present invention does not involve addition of liquid oxygen to the fermentation medium.

In yet an embodiment of the present invention the process of the present invention does not involve addition of gaseous oxygen and liquid oxygen to the fermentation medium.

In an embodiment of the present invention the oxygen present in the fermentation medium during the semi-aerobic fermentation process or the anaerobic fermentation process may be selected from bound oxygen, such as methanol; formic acid; formaldehyde or a derivative thereof.

The bound oxygen present in the fermentation medium during the semi-aerobic fermentation process or the anaerobic fermentation process may be, such as methanol; formic acid; formaldehyde or a derivative thereof may be formed in the fermentation reactor by the components added to the fermentation reactor or the bound oxygen, such as methanol; formic acid; formaldehyde or a derivative thereof may be added to the fermentation reactor.

The experimentally obtained carbon yield Y_{CH4,X} of biomass X (with formula CH_{1.8}O_{0.5}N_{0.2} and formula weight 24.6 g per C atom) on methane is 0.52/1=0.52 mol carbon per mol carbon from methane or 0.8 kg biomass/kg methane=1.75 m³ methane (1 atm, 0° C.) per kg produced biomass.

The traditional biomass production using microorganisms capable of metabolising methane under aerobic conditions may be collected in the following net stoichiometry:

$${CH}_{4} + 1.45 {O}_{2} + 0.104 {NH}_{3} \to 0.52 {CH}_{1.8} {O}_{0.5} {N}_{0.2} + 0.48 {CO}_{2} + 1.69 {H}_{2} O$$

The O₂ requirement is 1.75.1.45=2.54 m³ O₂ (1 atm, 0° C.) per kg biomass.

The large yield coefficient Y_{CH4}, O₂=1.45 for O₂ on CH₄ may be an economic burden on the production of biomass by the aerobic process. One would prefer to use atmospheric air (21% O₂), but now mass transfer of O₂ to the liquid phase (the fermentation medium) easily becomes limiting and the capital cost increases drastically. A compromise is to use technically pure O₂ (>90%) in the feed gas. Now mass transfer resistance is mostly avoided, but O₂ can become the economically dominant substrate.

Hence, the present invention provides significant advantages as a significant cost saving is obtained relative to the conventional processes, as the addition of oxygen is reduced, or even avoided, in the fermentation process of providing a biomass, such as single cell protein (SCP).

In addition, the heat of reaction also becomes a technical as well as an economic problem. As an empirical rule the heat of reaction in any aerobic stoichiometry is equal to 460×Y_{Cxx,O2} kJ where Cxx is the formula of the carbon source per C-mol carbon. In the above net stoichiometry, Y_{Cxx,O2}=Y_{CH4,O2}=1.45, and the heat release is 460×1.45×(1000/(24.6×0.52))=52159 kJ per 1 kg biomass produced. This much heat cannot be removed from even a small 50 to 100 m³ stirred tank reactor by cooling inside the tank.

One must either install large exterior refrigeration equipment, or the bioreactor must be long and slender, a design that makes a loop reactor almost the only rational choice.

The high value of Y_{Cxx,O2} in biomass fermentation with M. capsulatus has, indeed, been an ugly surprise for manufacturers of biomass, such as single cell protein (SCP).

Hence, the inventors of the present invention suggested to reduce oxygen in the fermentation process, even to reduce the oxygen as much as to provide a semi-aerobic fermentation process or even an anaerobic fermentation process to allow SCP production with methane as substrate has a far better stoichiometry than the above net stoichiometry:

$$0.525 {CH}_{4} + 0.475 {CO}_{2} + 0.2 {NH}_{3} \to {CH}_{1.8} {O}_{0.5} {N}_{0.2} + 0.45 {H}_{2} O$$

From this reaction the inventors of the present invention found that CH₄ may react with CO₂ in order to form formaldehyde and derivates thereof suitable for SCP production under semi-aerobic fermentation and anaerobic fermentation processes.

In an embodiment of the present invention the content of undissolved C1-C5 carbon source, e.g. methane, in the fermentation reactor or the content of gaseous C1-C5 carbon source, e.g. methane, present in the exhaust gas may be above 5% (vol/vol), such as at least 7.5% (vol/vol), e.g. at least 10% (vol/vol), such as at least 15% (vol/vol), e.g. at least 20% (vol/vol), such as at least 25% (vol/vol), e.g. at least 40% (vol/vol), such as at least 60% (vol/vol), e.g. at least 75% (vol/vol), such as at least 85% (vol/vol), e.g. at least 90% (vol/vol), such as at least 95% (vol/vol), e.g. at least 98% (vol/vol).

The fermentation process may further comprise a step of adding a nitrogen source to the fermentation reactor and/or to the inoculated fermentation medium. The Addition of the nitrogen source may be a continuous addition of the fermentation source.

In the present context, the term “continuous addition” relates to an ongoing addition of e.g. C1-C5 carbon source, e.g. methane, oxygen, CO₂ and/or nitrogen throughout the fermentation process depending on the need and/or based on the information received from one or more sensors of the fermentation reactor.

In an embodiment of the present invention the nitrogen source may be selected from ammonia, nitrate, molecular nitrogen, and a combination hereof. Preferably, the nitrogen source comprises ammonia.

Preferably, the ratio between C1-C5 carbon source, e.g. methane, and ammonia in the fermentation medium may be at least 2:1 (vol/vol), such as at least 3:1 (vol/vol), e.g. at least 5:1 (vol/vol), such as at least 10:1 (vol/vol), e.g. at least 12:1 (vol/vol), such as at least 14:1 (vol/vol), e.g. at least 16:1 (vol/vol), such as at least 18:1 (vol/vol), e.g. at least 20:1 (vol/vol), such as at least 25:1 (vol/vol), e.g. at least 30:1 (vol/vol).

In an embodiment of the present invention the mole ratio between C1-C5 carbon source, e.g. methane, and ammonia dissolved in the fermentation medium is at about 100:1, e.g. about 50:1; such as 10:1, e.g. about 3:1, such about 2:1, e.g. about 1:1.

In a further step of the cultivation process according to the present invention the biomass may be harvested.

Hence, the cultivation process according to the present invention may be continued until a biomass concentration of at least 1.0 g/L has been reached, such as a biomass concentration of at least 1.25 g/l, e.g. a biomass concentration of at least 1.5 g/l, such as a biomass concentration of at least 1.75 g/l, e.g. a biomass concentration of at least 2.0 g/l, such as a biomass concentration of at least 2.25 g/l, e.g. a biomass concentration of at least 2.5 g/l, such as a biomass concentration of at least 2.75 g/l, e.g. a biomass concentration of at least 3.0 g/l, such as a biomass concentration of at least 3.25 g/l, e.g. a biomass concentration of at least 3.5 g/l, such as a biomass concentration of at least 3.75 g/l, e.g. a biomass concentration of at least 4.0 g/l.

The inventors of the present invention found that the biomass produced according under semi-aerobic fermentation conditions or anaerobic fermentation conditions showed to have significant difference in the transcription level of several genes compared to the transcription level of biomass produced under aerobic fermentation conditions.

In an embodiment of the present invention the biomass material comprises at least 25 genes, such as at least 50 genes, e.g. at least 75 genes, such as at least 100 genes, e.g. at least 125 genes, such as at least 150 genes, e.g. at least 175 genes, such as at least 200 genes, e.g. at least 210 genes, such as at least 219 genes having a reduced transcript level of at least 25% (w/w), such as at least 50% (w/w), e.g. at least 60% (w/w).

The biomass material according to the present invention may be provided in liquid or solid form, such as in the form of a powder, a paste, a slurry, a capsule, or a sachet.

In an embodiment of the present invention the fermentation process may be a batch fermentation, a fed batch, or a continuous fermentation process. Preferably, the fermentation process may be a continuous fermentation process.

In a further embodiment of the present invention the continuous fermentation process may be conducted as a chemostat, pH-stat, productstat or other continuous fermentation process modes.

In an embodiment of the present invention the undissolved gas (such as oxygen and/or C1-C5 carbon source, e.g. methane and/or CO₂) comprises undissolved oxygen and/or undissolved C1-C5 carbon source, e.g. methane and/or undissolved CO₂ in the fermentation medium, in the top tank and in air pockets in the fermentation reactor.

The fermentation process according to the present invention may results in an improved biomass production and/or an increased growth rate of the microorganism.

In an embodiment of the present invention the method of the present invention provides a microbial growth rate during the fermentation process of at least 0.04 h⁻¹, e.g. at least 0.05 h⁻¹, such as at least 0.06 h⁻¹, e.g. at least 0.08 h⁻¹, such as at least 0.10 h⁻¹, e.g. at least 0.12 h⁻¹, such as at least 0.14 h⁻¹, e.g. at least 0.15 h⁻¹, such as at least 0.16 h⁻¹, e.g. at least 0.17 h⁻¹, such as at least 0.18 h⁻¹, e.g. at least 0.19 h⁻¹, such as at least 0.20 h⁻¹, e.g. at least 0.22 h⁻¹, such as at least 0.25 h⁻¹, e.g. at least 0.27 h⁻¹, such as at least 0.30 h⁻¹, e.g. at least 0.32 h⁻¹, such as at least 0.35 h⁻¹, e.g. at least 0.37 h⁻¹.

In another embodiment of the present invention a biomass production of at least 2.5 g/l on a dry-matter basis may be provided, such as a biomass production of at least 3.0 g/l on a dry-matter basis may be provided, e.g. a biomass production of at least 3.5 g/l on a dry-matter basis may be provided, such as a biomass production of at least 4.0 g/l on a dry-matter basis is provided, e.g. a biomass production of at least 7.5 g/l on a dry-matter basis may be provided, such as a biomass production of at least 10.0 g/l on a dry-matter basis may be provided, e.g. a biomass production of at least 20.0 g/l on a dry-matter basis may be provided, such as a biomass production of at least 30.0 g/l on a dry-matter basis may be provided.

The microorganism may be selected from the group consisting of bacteria, fungal, algae, and animal. Preferably, the microorganism may be a bacteria.

Preferably the one or more microorganism may be one or more aerobic microorganism.

In an embodiment of the present invention the one or more microorganism may be a methanotrophic microorganism or a methylotrophic microorganism.

In yet an embodiment of the present invention the methanotrophic microorganism may be a methanotrophic bacteria, preferably selected from a Methylococcus strain.

In an even further embodiment of the present invention the Methylococcus strain may be Methylococcus capsulatus.

In a further embodiment of the present invention the one or more microorganism may comprises a combination of M. capsulatus with one or more of A. acidovorans (preferably NCIMB 13287); B. firmus (preferably NCIMB 13289); and/or A. danicus (preferably NCIMB 13288).

In yet an embodiment of the present invention the fermentation process only comprises bacterial cells capable of metabolising methane, in particular only comprises microorganisms capable of metabolising methane.

In an embodiment of the present invention, the fermentation process only comprises microorganisms capable of metabolising methane under semi-aerobic conditions or anaerobic conditions.

In another embodiment of the present invention, the fermentation process does not comprise a co-culture comprising organisms that are not capable of metabolising methane, such as cyanobacterium. In particular, the semi-aerobic conditions or anaerobic fermentation process may not comprise a co-culture comprising organisms that are not capable of metabolising methane, such as cyanobacterium.

The fermentation process, in particular the anaerobic fermentation process, may comprise a single stain capable of metabolising methane.

When adding a gas, such as methane, CO₂ and/or oxygen, to a fermentation medium the gas will be undissolved. A natural mass transfer will occur of the undissolved gas to the fermentation medium and become a dissolved gas. It is the dissolved gasses that are capable of supporting growth of the microbial organisms during fermentation.

The process according to the present invention may preferably be conducted in a fermentation reactor as described in the present context.

A preferred embodiment of the present invention relates to a fermentation reactor for fermenting one or more microorganisms, the fermentation reactor comprising at least one inlet for introducing a C1-C5 carbon source into the fermentation reactor and one or more sensors, wherein the one or more sensors are one or more sensors for determining the concentration of undissolved and/or released gases in the fermentation reactor.

In an embodiment of the present invention the fermentation reactor may comprise a C1-C5 carbon source which is a C1 carbon source or a derivative thereof. The C1 carbon source may be methane; methanol, formic acid, formaldehyde or a derivative thereof.

In yet an embodiment of the present invention the at least one inlet for introducing the C1-C5 carbon source into the fermentation reactor is at least one gas inlet, preferably, the at least one gas inlet is a methane gas inlet.

In a further embodiment of the present invention the fermentation reactor does not comprise a vent for discharging undissolved and/or released gas(es) from the fermentation reactor.

In another embodiment of the present invention the fermentation reactor comprises a vent for discharging undissolved and/or released gas(es) from the fermentation reactor.

Preferably, the fermentation reactor may be an airlift reactor, a loop-reactor, a U-shape reactor, or a stirred tank reactor, preferably, the fermentation reactor is a loop-reactor or a U-shape reactor.

In an embodiment of the present invention the fermentation reactor is an airlift reactor, a loop-reactor, a U-shape reactor, or a stirred tank reactor (preferably, a loop-reactor or a U-shape reactor) wherein the fermenter (preferably the top tank) comprises at least one visual inspection means.

In an embodiment of the present invention the visual inspection means may be placed with a horizontal or substantial horizontal inspection view.

In a further embodiment of the present invention the visual inspection means may be placed on the side of the top tank allowing a combined view above the surface of a fermentation liquid and below the surface of the fermentation liquid.

Preferably, the visual inspection means may be placed in the end of the top tank.

Even more preferably, the visual inspection means may be placed in the end of the top tank providing a view from the first inlet (or the upflow part) towards the first outlet (or the downflow part).

In an embodiment of the present invention the visual inspection means may be an inspection hole, a camera, or a combination of an inspection hole and a camera.

Preferably, the inspection hole may be a sight glass.

The camera may be an inline camera.

In an embodiment of the present invention the top tank may be provided with a light source in order improve the visual inspection inside the top tank. The light source may be provided as a window allowing surrounding light to enter the top tank and/or as an artificial light source incorporated into the top tank.

The introduction of visual inspection means into the fermentation reactor (in particular ino the top tank of a loop-reactor or a U-shape reactor, may allow direct information and/or real time information on the foaming characteristics in the top tank. The foaming characteristics in the top tank (such as foaming density, foaming height, and level of turbulence provided in the top tank) may be indicative of proper C1-C5 carbon source to oxygen ratio and/or for evaluating the risk for explosion. Therefore, the inventors of the present invention found that foam sensors traditionally used, are not sufficient for surveilling the fermentation process.

A preferred embodiment of the present invention relates to a fermentation reactor for fermenting one or more microorganisms, the fermentation reactor comprising at least one gas inlet for introducing a methane gas into the fermentation reactor; and a vent for discharging undissolved and/or released gas(es) from the fermentation reactor, and one or more sensors, wherein the one or more sensors are one or more sensors for determining the concentration of undissolved and/or released gases in the fermentation reactor.

The present invention may also relate to a fermentation reactor for fermenting one or more microorganisms, the fermentation reactor comprising at least one inlet for introducing a C1-C5 carbon source, such as methane, into the fermentation reactor and one or more sensors, wherein the one or more sensors are one or more sensors for determining the concentration of undissolved and/or released gases in the fermentation reactor, wherein the undissolved and/or released gases is oxygen.

In a preferred embodiment of the present invention the fermentation reactor further comprises a vent for discharging undissolved and/or released gas(es) from the fermentation reactor.

In another embodiment of the present invention the fermentation reactor does not comprise a vent for discharging undissolved and/or released gas(es) from the fermentation reactor.

The fermentation reactor may preferably be an airlift reactor, a loop-reactor, a U-shape reactor, or a stirred tank reactor. Even more preferably, the fermentation reactor may be a loop-reactor or a U-shape reactor. Examples of providing a basic construction of the fermentation reactor, including sensors, mixers, pressurised zones and pumps, may be as described in WO 2010/069313 and/or WO 2000/70014, which are hereby incorporated by reference.

In an embodiment of the present invention the fermentation reactor may comprises one or more static mixers.

In a further embodiment of the present invention the fermentation reactor comprises one or more dynamic mixers.

The fermentation reactor according to the present invention, may comprise at least one static mixer and at least one dynamic mixer.

The fermentation reactor may further comprise a circulation pump.

The fermentation reactor according to the present invention may further comprises at least one gas inlet for introducing an oxygen gas into the fermentation reactor.

The fermentation reactor according to the present invention may further comprises at least one inlet for introducing CO₂ into the fermentation reactor.

The fermentation reactor according to the present invention may even further comprise at least one inlet for introducing a nitrogen source into the fermentation reactor.

In an embodiment of the present invention the one or more sensors are one or more sensors for determining the concentration of undissolved and/or released oxygen in the fermentation reactor. The one or more sensors may be one or more sensors for determining the concentration of undissolved and/or released methane in the fermentation reactor.

In the event the fermentation reactor is to be used for an anaerobe fermentation process there would be no requirements for the microbial organisms for the presence of oxygen. Hence, in an embodiment of the present invention the fermentation reactor does not comprise a gas inlet for introducing an oxygen gas into the fermentation reactor.

The fermentation reactor according to the present invention may further comprises a CO₂ sensor.

In an embodiment of the present invention the fermentation reactor may further comprises a nitrogen sensor.

In an embodiment of the present invention the at least one sensor may be placed an area of the fermentation reactor flooded with fermentation medium.

In a further embodiment of the present invention the at least one sensor may be placed next to the vent. The at least one sensor may be placed in the top tank.

The at least one gas inlet for introducing an oxygen gas into the fermentation reactor may be controlled by a computer.

The at least one gas inlet for introducing a methane gas into the fermentation reactor may be controlled by a computer.

The one or more sensors may be controlled by a computer.

In an embodiment of the present invention the at least one gas inlet for introducing an oxygen gas into the fermentation reactor and/or the at least one gas inlet for introducing a methane gas into the fermentation reactor may be controlled by a response obtained from the one or more sensors. Preferably, this control is performed by a computer.

An aspect of the present invention relates to a feed product for an animal or an ingredient for a feed product for an animal comprising the biomass material of the present invention; or a fish feed product or an ingredient of for a fish feed product comprising the biomass material according to the present invention; or a human food product or an ingredient for a human food product comprising the biomass material according to the present invention.

In an embodiment of the present invention the feed product for an animal or the ingredient for a feed product for an animal; or the fish feed product or the ingredient of for a fish feed product; or the human food product or the ingredient for a human food product may be in dried form or in liquid form.

It should be noted that embodiments and features described in the context of one of the aspects of the present invention also apply to the other aspects of the invention.

All patent and non-patent references cited in the present application, are hereby incorporated by reference in their entirety.

### EXAMPLES

Example 1—Anaerobic Fermentation of Methanotrophic Bacteria.

A continuous fermentation of M. capsulatus was performed in a pilot plant and was running with 10 g/L in the continuous phase where a lot of Nitrate/Nitrite accumulated and after continuous fermentation supply of oxygen was stopped and no introduction of oxygen has been stopped. Supply of CO₂ was started into the U-loop reactor in order to provide a completely anaerobic phase in the U-loop reactor.

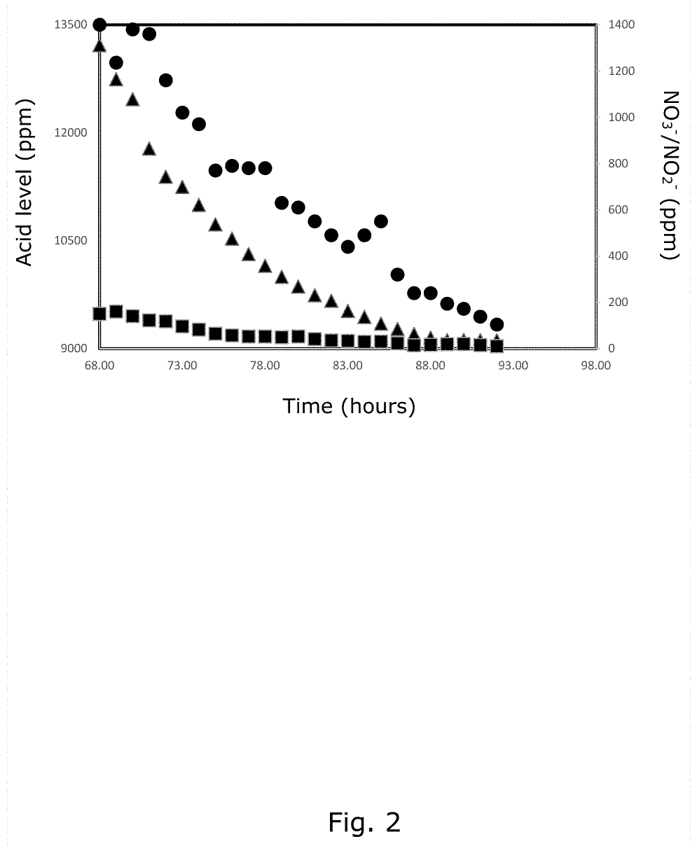

FIG. 2 shows the growth of methanotrophs (M. capsulatus) under anaerobic conditions for 92 hours at continuous fermentation in the pilot plant. After fermenting for 68 hours the supply of oxygen was stopped and the continuous growth of the biomass may be followed by the reduction in the acid level (indicated by triangles); the NO₂⁻ level (indicated by squares) and the NO₃⁻ level (indicated by circles), which all drops significantly over a period of 24 hours (from 68 hours from the start of continuous fermentation to 92 hours). Hence, anaerobic fermentation of methanotrophs, such as M. capsulatus may be performed without the supply of gaseous oxygen.

The biomass showed continuous growing slowly up to 11.5 g/L by consuming acid, nitrate (from 1400 ppm to 100 ppm) and nitrite (from 150 ppm to 10 ppm) present in the fermentation medium over a period of 24 hours.

This clearly demonstrates that within just 24-hour the biomass was able to grow under anaerobic fermentation conditions without any undissolved oxygen in the fermentation reactor and/or a content of gaseous oxygen in an exhaust gas.

The resulting biomass was analysed as described by Linde et al (1999) and the biomass showed to have decreased transcription level of at least 75 genes compared to the transcription level of biomass produced under aerobic fermentation conditions which was due to the anaerobic fermentation process.

### REFERENCES

WO 2010/069313
WO 2000/70014
Linde et al (1999); “Genome-wide transcriptional analysis of aerobic and anaerobic chemostat cultures of Saccharomyces cerevisiae”; J. Bacteriology, December 1999, Vol. 181, No. 24, p: 7409-7413

1. Process for cultivating one or more microorganisms capable of metabolising methane, the process comprises the steps of:
(i) adding a fermentation medium to a fermentation reactor;
(ii) adding the one or more microorganism to a fermentation reactor, providing an inoculated fermentation medium, wherein the one or more microorganism does not include a recombinant microorganism;
(iii) adding a C1-C5 carbon source, e.g. methane, to the fermentation reactor and/or the inoculated fermentation medium during the fermentation of the one or more microorganisms; and
(iv) optionally, adding oxygen to the fermentation reactor and/or the inoculated fermentation medium during the fermentation of the one or more microorganisms,
wherein the process is a semi-aerobic fermentation process or an anaerobic fermentation process wherein the content of undissolved oxygen in the fermentation reactor or the content of gaseous oxygen present in an exhaust gas may be at most 5% (vol/vol).

2. The process according to claim 1, wherein the semi-aerobic fermentation process may be a fermentation process wherein the content of undissolved oxygen in the fermentation reactor or the content of gaseous oxygen present in an exhaust gas is 0% (vol/vol).

3. The process according to anyone of claims 1-2, wherein the content of undissolved CO₂ in the fermentation reactor or the content of gaseous methane present in the exhaust gas is above 2.5% (vol/vol), such as at least 4% (vol/vol), e.g. at least 5% (vol/vol), such as at least 7.5% (vol/vol), e.g. at least 10% (vol/vol), such as at least 12.5% (vol/vol), e.g. at least 15% (vol/vol), such as at least 20% (vol/vol), e.g. at least 30% (vol/vol), such as at least 40% (vol/vol), and/or the content of undissolved CO₂ is less than 75% (vol/vol), such as less than 70% (vol/vol), e.g. less than 60% (vol/vol), such as less than 50% (vol/vol), e.g. less than 40% (vol/vol), such as less than 30% (vol/vol), e.g. less than 20% (vol/vol), such as less than 10% (vol/vol), e.g. less than 5% (vol/vol).

4. The process according to anyone of claims 1-3, wherein the one or more microorganism is one or more aerobic microorganism.

5. The process according to claim 4, wherein the one or more aerobic microorganism is one or more aerobic methanotrophic microorganism and/or or one or more aerobic methylotrophic microorganism.

6. The process according to claim 5, wherein the one or more aerobic methanotrophic microorganism or one or more aerobic methylotrophic microorganism is one or more aerobic methanotrophic bacteria and/or one or more aerobic methylotrophic bacteria.

7. The process according to claim 6, wherein the one or more aerobic methanotrophic bacteria is selected from a Methylococcus. Preferably, M. capsulatus.

8. The process according to anyone of claim 6 or 7, wherein the one or more aerobic methanotrophic bacteria comprises a combination of M. capsulatus (preferably, NCIMB 11132); A. acidovorans (preferably NCIMB 13287); B. firmus (preferably NCIMB 13289); and A. danicus (preferably NCIMB 13288).

9. A fermentation reactor for fermenting one or more microorganisms, the fermentation reactor comprising at least one inlet for introducing a C1-C5 carbon source, such as methane, into the fermentation reactor and one or more sensors, wherein the one or more sensors are one or more sensors for determining the concentration of undissolved and/or released gases in the fermentation reactor, wherein the undissolved and/or released gases is oxygen.

10. The fermentation reactor according to claim 9, wherein the fermentation reactor further comprises a vent for discharging undissolved and/or released gas(es) from the fermentation reactor.

11. The fermentation reactor according to claim 9, wherein the fermentation reactor does not comprise a vent for discharging undissolved and/or released gas(es) from the fermentation reactor.

12. The fermentation reactor according to anyone of claims 9-11, wherein the fermentation reactor is an airlift reactor, a loop-reactor, a U-shape reactor, or a stirred tank reactor, preferably, the fermentation reactor is a loop-reactor or a U-shape reactor.

13. A biomass material obtainable by the process according to anyone of claims 1-8.

14. The biomass material according claim 13, wherein the biomass material comprises at least 25 genes having a reduced transcript level of at least 25% (w/w).

15. A feed product for an animal; or a fish feed product; or a human food product comprising the biomass material according to anyone of claims 13-14.
